(19) United States
(12) Patent Application Publication
(10) Pub. No.: US 2017/0027773 A1
(43) Pub. Date: Feb. 2, 2017

(54) POCKET POUCH

(71) Applicant: Patricia Elizabeth Morgan, Upper Marlboro, MD (US)

(72) Inventor: Patricia Elizabeth Morgan, Upper Marlboro, MD (US)

(21) Appl. No.: 15/097,549

(22) Filed: Apr. 13, 2016

Related U.S. Application Data
(60) Provisional application No. 62/282,277, filed on Jul. 30, 2015.

(51) Int. Cl. A61F 13/551; B65D 65/14; B65D 71/06; A61F 13/84

(52) U.S. Cl. CPC A61F 13/5519; A61F 13/84; A61F 13/5511; A61F 13/5512; B65D 65/14; B65D 71/06

(57) ABSTRACT

Provided herein is a Pocket Pouch comprising one or more absorbancy layers; a moisture-free liner; and one or more re-sealable strips. Also provided herein are methods for attaching the Pocket Pouch to a cloth or disposable diaper, training pant, disposable bladder leak underwear, sanitary napkins, and/or disposable bib.

### PRIORITY

This application claims priority to U.S. Provisional Application No. 62/282,277, which was filed on Jul. 30, 2015, the contents of which is incorporated herein by reference in its entirety.

### BACKGROUND OF THE INVENTION

The invention described herein relates to an item of convenience for a variety of individuals and service providers, including but not limited to: parents; daycare providers; healthcare providers; hospitals; in-home care; assisted living; individuals who use sanitary napkins and/or disposable bladder leak underwear; restaurants or other establishments that offer bibs to their patrons (e.g., Red Lobster, Olive Garden); and companies that provide clothing for animals and humans.

Parents, Providers and/or Servers in these industries are required to gather multiple items at a time, requiring the use of both hands and loss of time. For example, when performing a diaper change or packing a diaper bag, the provider has to gather the diapers, petroleum jelly, wipes and a disposable bag. Pulling all of this together becomes inconvenient and providers on the go often forget to grab one or more necessities. The invention provided herein alleviates these issues.

### SUMMARY OF THE INVENTION

Provided herein is a Pocket Pouch, which includes: moisture-free lining, re-sealable strips (e.g., strips that include an adhesive; Velcro or other means to secure the contents); and the contents required for the particular task being accomplished. For example, in some non-limiting embodiments of the invention, a Pocket Pouch for changing a diaper could contain: individual wipes; a 1-5 oz container of petroleum jelly; a 1-5 oz container of lotion; a 1-5 oz container of baby powder; and a disposable bag all attached to the disposable diaper.

In some embodiments, the Pocket Pouch and its contents are attached to a cloth diaper; training pant; or disposable bib. In various embodiments, the Pocket Pouch contains two or more of the individual contents to allow for multiple changes.

In specific embodiments, the Pocket Pouch for changing a diaper contains: 2 individual wipes; a 3″×3″package wipe size 8″×6″; 5 oz. container of petroleum jelly; 5 oz container of lotion; 5 oz. container of container of baby powder; and a disposable bag all attached to the disposable diaper.

In various embodiments, the Pocket Pouch varies in shape, size, colors, and type of material (recyclables) depending on the Embodiment.

In some embodiments, the contents of the Pocket Pouch are hypoallergenic; fragrance and alcohol free; sensitive; ecologically friendly; and easy to travel with.

Accordingly, the invention described herein affords greater convenience to parents; providers; servers and other individuals to have at hand everything needed in a single easy to use pouch. This allows for easy travel, no worries about running out of sanitary condiments, and the eco-friendly safe disposal of soiled items in private or public environments.

### BRIEF DESCRIPTION OF THE DRAWINGS

FIG. 1 and FIG. 2 illustrate the Pocket Pouch attached to the interior/back of disposable diaper with a moisture-free lining and re-sealable strips, according to these embodiments of the invention.

FIG. 3 illustrates an embodiment of the Pocket Pouch attached to the interior/back of training pant with a moisture-free lining and re-sealable strips, according to one embodiment of the invention.

FIG. 4 illustrates an embodiment of the Pocket Pouch attached to the disposable bib through the use of adhesive shoulder straps for easy application and removal with a moisture-free lining and re-sealable strips, according to one embodiment of the invention.

The details of one or more embodiments of the invention are set forth in the accompanying description below. Other features, objects, and advantages of the invention will be apparent from the description and from the claims.

### DETAILED DESCRIPTION

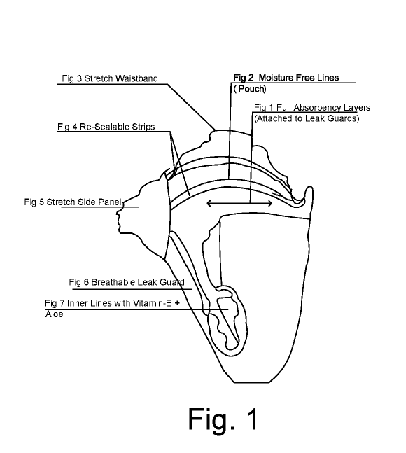

In accordance with one aspect of the invention, FIG. 1 illustrates an embodiment of the Pocket Pouch attached to the interior/back of disposable diaper. In this embodiment, the Pocket Pouch (1) includes: hypoallergenic absorbency layers (1A); a full absorbency layer attached to leak guards (2); a moisture free liner 3½ inches deep (3); a stretch waistband (4); re-sealable strips (5); a soft stretch side panel(s) (6); soft, breathable stretch leak guards (7); and a soft inner liner with vitamin E and aloe integrate from the front of the diaper to the back of the diaper. In other embodiments, the Pocket Pouch is made as an individual pouch. Depending on the use and preference of the user, embodiments of the Pocket Pouch vary in shape, size, color and type of material.

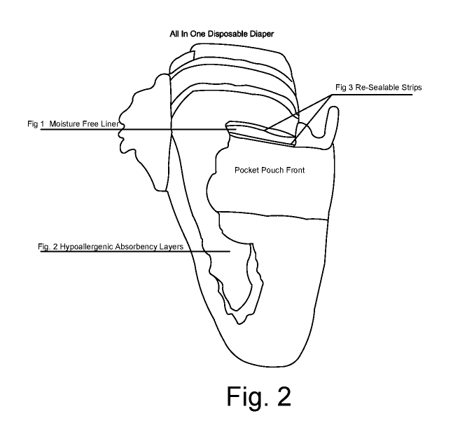

In accordance with one aspect of the invention, FIG. 2 illustrates an All In One disposable diaper. In this embodiment, the disposable diaper Pocket Pouch includes a right pocket opening to a moisture-free liner (1); hypoallergenic absorbency layers (2); re-sealable strips (3); and absorbency layers (4). In this illustrative embodiment, the Pocket Pouch is located on the Interior/Front of the disposable diaper. There are two (2) Absorbency Layers, integrated in the diaper, from the interior/back to the interior/front of the diaper with a moisture-free lining that stows the sanitary articles and re-sealable strips to secure the contents and to form the Pocket Pouch in the front of the diaper. The re-sealable strips can be made from made of adhesive, Velcro, or any other suitable fastening material/means known in the art.

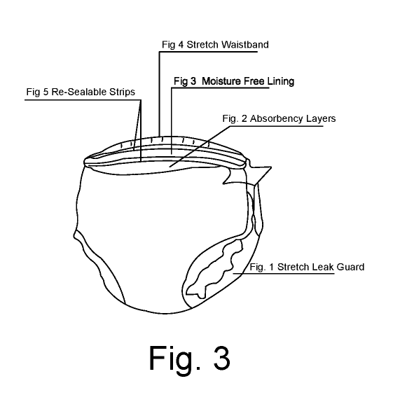

In accordance with another aspect of the invention, FIG. 3 illustrates an embodiment of the Pocket Pouch attached to the interior/back of training pant. This embodiment includes the Pocket Pouch (1, 1A); a moisture free lining (2); re-sealable strips (3); a soft stretch waistband (4); and a soft stretch leak guard (5).

In various embodiments, the content of the Pocket Pouch for Disposable Diapers and Training Pant is (2) 3″×3″ individually packaged Baby Wipes (wipe size 8″×6″), (1) 1-5 oz pack petroleum jelly, (1) 3″×3″ (bag size 8″×6″ sealable (black or dark color) disposable bag (fold size)3″×3″ (bag size 8″×6″), The No Strings Attached Disposable Bibs (2) 3″×3″ individually packaged Hand Wipes (wipe size 8″×6″), (1) 1-5 oz pack Lotion and Disposable Leak Underwear, Sanitary Napkins etc. (2) Vaginal Wipes and Disposable Bag. The contents of the Pocket Pouch are Hypoallergenic, Alcohol and fragrance—Free, Sensitive, Eco-Friendly, and Easy Travel.

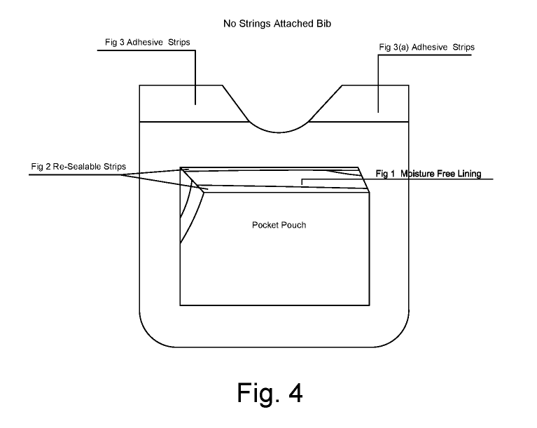

In accordance with yet another aspect of the invention, FIG. 4 illustrates an embodiment of the Pocket Pouch attached to a disposable bib through the use of adhesive straps. This embodiment includes a moisture resistant Pocket Pouch (1); re-sealable strips (2); and adhesive strips (3, 3A).

Provided in the illustrated embodiments, the Pocket Pouch that holds or stows the contents is secured by Re-sealable Strips, Tape, Velcro or other attachment means know in the art. It should be understood that the location of the Pocket Pouch has been provided for illustrative purposes.

In further embodiment, the Bib is attached on the human at the shoulders using adhesive, Velcro or other. The Pocket Pouch also includes, Re-sealable Strips (to open and close pouch) to secure stowage in a moisture-free lining. The Pocket Pouch can also be embodied on the Exterior Front and Back of the Diaper, Training Pant, or other.

In further embodiment, said Pocket Pouch embodied in the diaper, training pant, bib or other provides, stowage for everything needed for a diaper change etc., ALL IN ONE POUCH. Making life easier, not having to gather each item, worry about running out of wipes, petroleum jelly etc., but more specifically The Pocket Pouch allows an individual, peace of mind, at home, work, in public, or private, and allows for easy travel.

Other aspects, features, and embodiments of the invention will become apparent upon review of the illustrative embodiments, of the accompanying drawings. The invention though is pointed out with particularity by the appended claims.

While the foregoing specification teaches the principles of the present invention, and specific embodiments of the invention have been described for the purposes of illustration, and examples have been provided for the purposes of illustration, it will be understood that various modifications may be made without deviating from the spirit and scope of the invention as come within the scope of the invention.

1. A Pocket Pouch comprising:
a. one or more absorbancy layers;
b. a moisture-free liner; and
c. one or more re-sealable strips.

2. The Pocket Pouch of claim 1, wherein the re-sealable strips are made of adhesive, tape or velcro or the disposable pocket pouch bib.

3. The Pocket Pouch of claim 1, wherein the Pocket Pouch is embroided or attached on or to the exterior or interior of a cloth or disposable diaper, training pant, disposable bladder leak underwear, sanitary napkins, disposable underwear with sanitary napkin, or disposable bib.

4. The Pocket Pouch of claim 1, further comprising a clip capable of securing the Pocket Pouch to a waistband or a pocket.

5. The Pocket Pouch of claim 1, further comprising a strap capable of securing the Pocket Pouch over the shoulder of a user.

6. The Pocket Pouch of claim 2, wherein the Pocket Pouch holds individually wrapped wipes, disposable bags and/or other sanitation items.

7. The Pocket Pouch of claim 6, wherein the Pocket Pouch holds a 1-5 oz container of petroleum jelly and/or a 1-5 oz container of lotion.

8. The Pocket Pouch of claim 6, wherein the Pocket Pouch holds a 5 oz container of petroleum jelly and/or a 5 oz container of lotion.

9. The Pocket Pouch of claim 1, wherein the material used to make the Pocket Pouch is ecological friendly and/or biodegradeable.
